(19) United States
(12) Patent Application Publication
(10) Pub. No.: US 2010/0234224 A1
(43) Pub. Date: Sep. 16, 2010

(54) COMPOSITIONS OF INCREASING MICROBIAL POPULATIONS ON SURFACES AND THEIR USES

(76) Inventor: Dal Soo Kim, Seoul (KR)

Correspondence Address: ROBERT E. BUSHNELL & LAW FIRM, 2029 K STREET NW, SUITE 600, WASHINGTON, DC, 20006-1004, US

(21) Appl. No.: 12/734,021

(22) PCT Filed: Sep. 26, 2008
(86) PCT No.: PCT/KR2008/005701

§ 371 Date: Apr. 5, 2010

(30) Foreign Application Priority Data
Oct. 5, 2007 (KR) 10-2007-0100278

(51) Int. Cl. A01N 63/00; C08B 31/00; C12N 1/20; A01P 7/04; A01P 13/00

(52) U.S. Cl. 504/117; 536/102; 435/253.6; 424/93.4; 424/93.46; 424/93.43

(57) ABSTRACT

The present invention relates to compositions that can improve biological activity or can increase population density of natural or beneficial microorganisms on surfaces. By proper use of the compositions, the present invention provides uses of protecting plants from plant pathogen, harmful insects or weedy plants or of promoting plant growth. By proper use of the compositions in the present invention, biological activity or population density of natural or beneficial microorganisms can be increased and the improved biological activity or population density of the microorganisms can protect plants from harmful organisms including plant pathogens, harmful insects or weedy plants or can promote plant growth.

### TECHNICAL FIELD

The present invention relates to compositions to increase biological activity or population density of microorganisms on surface. Compositions of the present invention can be used to protect plants from harmful organisms such as plant pathogens, insects or weedy plants, or to promote plant growth.

### BACKGROUND ART

There are various natural microorganisms living on plant surfaces including leaf, stem, branch, flower or fruit, on soil surface of lawn, or surfaces of an animal such as cockroaches, snails or ants (hereafter referred as “surfaces”). These microorganisms exist together with saprophytic, symbiotic or parasitic interactions. On plant surfaces, parasitic plant pathogens cause plant diseases usually by increasing their populations under favorable environmental conditions, and harmful insects also increase their population resulting in damages to plants. Weed plants also results in reduced growth of cultivating crops or plants due to competition among plants for nutrition or space. The plant pathogens, harmful insects and weeds (hereafter referred as “harmful organisms”) have been threats of human efforts to secure stable production of foods for mankind. Currently, humans mainly rely on use of chemical pesticides to protect plants from these harmful organisms. Up to now, chemical pesticides have been used as the most effective means for protecting crops from plant pathogens, insects and weeds. Current market size is approximately 30 US billion dollars in the world and 1 US billion dollars in Korea. However, chemical pesticides are known with many adverse problems of causing ecological disorders by suppressing not only harmful organisms but also other natural beneficial organisms as an adverse side effect. Residues or metabolites of chemical pesticides may also have a toxic effect to producers of chemical pesticides, farmers or consumers. In addition, repeated use of chemical pesticides may induce resistance to plant pathogens, insects or weeds, resulting in their reduced sensitivity to chemical pesticides. Sometimes, leakage of chemical pesticides may cause air, water or soil contamination of our environment.

Human efforts have been keeping to supplement or to replace chemical pesticides that have potential risks as described above. One alternative is use of certain microorganisms to manage harmful organisms. These microorganisms (hereafter referred as “plant protection microorganisms”) have their biological activity to inhibit certain harmful organisms under favorable environment conditions for their growth. This microbial method has been socially needed mainly due to its safety profile to mammals and environments in comparison to chemical pesticides. Recently, many countries including the United States, European countries, Japan and Korea have launched regulations to encourage research, development and registration of industrial products of “plant protection microorganisms” to manage “harmful organisms”. “Plant protection microorganisms” are developed usually by being isolated from nature, artificially cultivated in their own media and formulated before use. However, this microbial method also has technical limitations and has not been successful in industrial viewpoints compared to chemical pesticides due to the technical limitations, although it is known to be safe to humans and environment. The microbial method is usually not sufficient to protect plants from “harmful organisms” or highly variable in its efficacy that highly depends on environmental conditions. Another limitation is relatively higher cost of microbial products, compared to chemical pesticides. “Plant protection microorganisms” usually require stricter conditions for their survival or biological activity during manufacturing, transportation, storage or after application by farmers in fields, and requirement of these strict conditions often results in higher costs than chemical pesticides. Especially, unstable or unreliable efficacy depending on environmental conditions often requires growers of taking economic risks from damages by “harmful organisms”. These problems are considered main reasons why microbial method has not been adopted widely in industry, although it is generally known to be safe to humans and environment.

People have been using chemical fertilizers for long time to enhance agricultural productivity. However, repeated use of chemical fertilizers has caused reduced productivity of agricultural land by salt accumulation in soils and other negative problems such as water pollution. In order to overcome these problems, people have been trying to recover productivity of agricultural land by using natural organic materials instead of chemical fertilizers. Natural organic materials are known to exhibit fertilizer effect by their metabolites including small organic molecules and minerals. These organic materials are mainly degraded by natural microorganisms. These microorganisms (hereafter referred as “plant growth promotion microorganisms”) exist in nature in a great amount, and proper use of these microorganisms can replace or supplement chemical fertilizers. However, “plant growth promotion microorganisms” have similar technical limitations as in the microbial method with “plant protection microorganisms” such as lower efficacy, unstable or unreliable efficacy by being sensitive to environment conditions, higher price than chemical fertilizers. These technical limitations also lead to limited use of microbial method in industrial applications.

The above-described unstable or insufficient effect of the methods based on “plant protection microorganisms” and “plant growth promotion microorganisms” (hereafter referred as “beneficial microorganism”) is caused by the fact that biological activities of plant protection or growth promotion on plant surfaces often largely depends on environmental conditions for their survival. More specifically, biological activities of microorganisms on plant surface are known to be affected by factors such as moisture, temperature, UV light, nutrients and so on (Steven E. Lindow and Maria T. Brandl. 2003. Microbiology of the phyllosphere. Applied and Environmental Microbiology 69:1875-1883). Beneficial microorganisms applied to plant surface for plant protection or plant growth promotion are also generally affected for their survival or biological activities by these environmental factors. Especially, under environment conditions that cause dryness of plant leaf and then reduced availability of nutrients in leaf or that expose plant leaves direct to sunlight, population of these beneficial organisms significantly decreases or their biological activities are lowered.

The present invention was focused on developing compositions for strengthening availability of water and microbial nutrients on plant surfaces, and therefore resulting in increased population or biological activities of microorganisms on surfaces. Water on plant surfaces is provided by natural precipitation or artificial irrigation, and water amount is fluctuating on plant surfaces in terms of time and space. Generally microorganisms are more sensitive to water for their biological activities than plants. More specifically, water on plant surface is dropped off leaves down to ground surface by gravity or vanished from plant surfaces by evaporation. And water remaining on plant surfaces is also variably distributed by surface structures on leaves. For example, water remaining on plant surfaces is mainly found along leaf veins or around trichomes, and results in uneven distribution of water that leads limited availability of water to microorganisms living on leaves. This may be one of reasons why microorganisms are found along leaf veins or around trichomes. Limited water availability for microorganisms (hereafter referred as “available water”) on plant surfaces generally leads to reduced microbial population or reduced biological activity and transformation into resting structures like spores. Therefore, “available water” is considered highly important to increase or maintain microbial populations or their biological activities. Furthermore, “available water” is also important for availability of microbial nutrients on surfaces to be accessible to microorganisms on plant surfaces. Microorganisms usually do not have means for active movement on plant surfaces and thus tend to get moved passively by water stream existing on plant surfaces. Therefore, limited water availability also means limited availability of microbial nutrients that are dissolved or suspended in water. As such, “available water” also plays a role to deliver microbial nutrients to microorganisms on plant surfaces.

Among microbial nutrients, carbon and nitrogen sources are utmost important for microorganisms to maintain their population or to express their biological activities. Generally, within a certain space and time point, various natural microorganisms exist on plant surface in a great amount. This coexistence of various microorganisms results in high competition among microorganisms for microbial nutrients (Steven E. Lindow and Maria T. Brandl. 2003. Microbiology of the phyllosphere. Applied and Environmental Microbiology. Vol 69:1875-1883). Especially, “beneficial microorganisms” that are introduced artificially by human for plant protection or plant growth promotion are usually positioned unfavorable for their survival or biological activities on plant surfaces that are already occupied by natural microorganisms with superior ecological fitness. Most “beneficial microorganisms” are grown artificially in culture media to optimize their maximum growth in population and then introduced to natural field conditions. Because of this reason, these “beneficial microorganisms” cannot be considered positioned competitive in natural ecological conditions. In addition, these “beneficial microorganisms” are generally manufactured in their resting structures like spores that are biologically less active than their vegetative structures. These manufacturing processes of growing them in artificial culture media and storing in resting structures can be the reasons of low competitiveness of “beneficial microorganisms” on plant surfaces compared to natural microorganism that are ecologically already fit for their survival.

Natural microorganisms that live on plant surfaces compete with plant pathogenic microorganisms for water, microbial nutrients or space, and this competition may result in reduced plant disease development. Therefore, increased competition between natural saprophytic microorganisms and plant pathogenic microorganisms can be considered desirable in terms of plant protection from plant pathogens. Moreover, in order to protect certain plants from certain plant pathogens, use of certain microorganisms that are selected for the competition, artificially cultured on media and to have increased population can be more desirable than simple increase of natural saprophytic microorganisms. On the other hand, plant protections from harmful insects or weedy plants by using natural microorganisms are fairly difficult because their population density or biological activity is not high enough to inhibit harmful insects or to suppress weedy plants. In this case, beneficial microorganisms that are artificially grown in culture media are more desirable. Therefore, certain beneficial microorganisms that specifically inhibit certain harmful insects or specifically inhibit certain weeds are needed to be artificially introduced to plant surfaces. For promotion of plant growth by using beneficial microorganisms, artificial introduction is desirable than use of natural saprophytes.

It has not been technically well established to provide microbial nutrients that can be used selectively by beneficial microorganisms but not by natural microorganism population. In nature, microorganisms on plant surfaces keep dynamically changing their species and numbers in given times and spaces. On plant surfaces, it is known that many different microorganisms exist together simultaneously. For example, 37 genera and 85 species of microorganisms are reported in phyllosphere of oat, olive, sugarcane and wheat (Ching-Hong Yang, David E. Crowley, James Borneman and Noel T. Keen. 2001. Microbial phyllosphere populations are more complex than previously realized. PNAS. Vol. 98:3889-3894). Such diversity of microorganisms may be similarly applied to other plants. Therefore, when beneficial microorganisms are artificially introduced on plant surfaces, they have to compete with other natural microorganisms within the same space under various environmental conditions. This dynamic competition makes beneficial microorganisms difficult to occupy a dominant position in given spaces for long time. One method to overcome this limitation can be use of microbial nutrients that are selectively used by certain beneficial microorganisms. For example, certain beneficial microorganisms can use oils as their carbon sources, whereas most natural microorganisms cannot use oils as nutrients. Another method is use of antimicrobial agents that are not inhibitory to one or some of beneficial microorganisms but inhibitory to most natural microorganisms. With this method, beneficial microorganisms can establish their population securely in their early application stage while natural microorganisms are selectively controlled. If needed, beneficial microorganisms can be induced to be resistant to certain antimicrobial agents in the laboratory and may be used for this purpose. Some chemical bactericides or fungicides can be used as the antimicrobial agents. However, even in this case, these antimicrobial agents are used at much lower application rates, due to the different purpose of their use and low use concentration.

A key concept of the present invention is to use super absorbent polymer (hereafter referred as “SAP”) to increase population density or biological activity of microorganisms on plant surfaces. SAP in the present invention is defined as all polymers that have hydrophilic function and that can hold tap water at least for several ten times of their own weight for a certain period of time. SAP was introduced by USDA in 1970s and industrially used since 1980s. Their main applications are for sanitation purposes including disposable diapers and hygienic bands for women. In agriculture and forestry, SAP also has been used in soil to provide plant roots with water for longer time in arid area or to increase aeration in soil (Sang-Bum Park. 1994. Characteristics of Super Absorbent Polymer and State of the Art. Mokchae Konghak 22(1): 91-112). Although SAP can be classified into natural and synthetic SAP based on source of its raw skeleton materials, it was obtained by crosslinking soluble raw polymer materials into non-soluble forms, which consequently can hold water several ten times of water of its own weight in a gel form. (Sang-Bum Park. 1994. Characteristics of Super Absorbent Polymer and State of the Art. Mokchae Konghak 22(1):91-112).

U.S. Pat. No. 9,009,020 describes manufacturing process of SAP for agricultural application. It is disclosed in this patent that SAP can be used in soil, on seed or root to improve aeration in soil, to improve germination and emergence of seeds, to improve plant growth and yield, and to reduce cost of water irrigation. For this purpose, inventors describe convenient use by manufacturing SAP having the size in range of 5-24 meshes. Korean Patent Application Laid-Open No. 2002-0002852 describes use of SAP on seeds pelleted with ectomycorrhizae to increase storage period, help germinated root to come out of epidermis of pelleted seeds and to reduce water stress of plant seedlings. Another research was conducted in Korea using 2% SAP of soil by weight in potting soil to improve suppression activity of Serratia plymutica A21-4 that is effective against soil born plant disease. By this application, emergence and growth promotion of red-pepper seedlings were reported to be significantly enhanced (Shun-Shan Shen, Won-il Kim and Chang-Seuk Park. 2006). Effect of hydrogel on survival of Serratia plymuthica A21-4 in soils and plant disease suppression. Plant Pathology Journal Vol. 22:364-368). In addition, population density of Serratia plymutica A21-4 was reported to be significantly increased on roots and rhizosphere whereas population density of Phytophthora capsici, a causal pathogen of red-pepper blight, was reduced.

However, all of these reports are directed to the use of SAP in soil that is working under soil surface. There has been no report about the use of SAP that is applied on plant surfaces to increase population density or biological activities of natural or beneficial microorganisms. In addition, there has been no report of using SAP to protect plants from harmful microorganisms or to promote plant growth by increasing population density or biological activities of microorganisms on plant surfaces.

### DETAILED DESCRIPTION OF THE INVENTION

### Technical Problem

The present invention was based on an idea of protecting plants from harmful organisms or promoting plant growth by increasing population densities or biological activity of saprophytic or beneficial microorganisms on plant, soil and organism surfaces, in combination with SAP to increase water availability needed for microorganisms on surfaces. Therefore, the present invention provides compositions containing SAP (hereafter referred as “SAP composition”) which can be used for increasing population densities or biological activity of microorganisms on surface, and can be used to protect plants from harmful organisms, to promote plant growth or others.

### Technical Solution

The present invention provides SAP compositions to increase microbial population or biological activities on plant surfaces. Also the present invention describes applications of SAP compositions on plant surfaces including leaves, stems, branches, flowers and fruits, soil surface where grasses grow, or animal surfaces of snails, cockroaches or ants. Using SAP compositions, the present invention describes how effectively SAP compositions can protect plants from harmful organisms or promote plant growth.

### ADVANTAGEOUS EFFECTS

SAP compositions of the present invention may provide additional water or microbial nutrients needed for natural or beneficial microorganisms living on surfaces of plants, soil and animals. This additional water or microbial nutrients may increase microbial population on surfaces and improve biological activity of the microorganisms. More specifically, the SAP compositions of the present invention provide additional water or microbial nutrients that extend supplying time of water or microbial nutrients to increase microbial population on surfaces. SAP compositions added with microbial nutrients are more effective to increase microbial populations on surfaces. The SAP compositions of the present invention, which comprise SAP, microbial nutrients and specific beneficial microorganisms, are prepared to increase the population of artificially-introduced beneficial microorganisms rather than natural saprophytic beneficial microorganisms. When the SAP compositions comprise beneficial microorganisms that are inhibitory to growth of natural saprophytic microorganisms, these SAP compositions may inhibit natural microorganisms, thus resulting in reduction of population density of the natural microorganisms. In addition, by adding some specific beneficial microorganisms that are selectively growing on specific microbial nutrients or that are resistant to certain specific antimicrobial agents in the SAP compositions, the SAP composition of the present invention can be used to increase specific beneficial microorganisms rather than natural saprophytic beneficial microorganisms.

In summary, the SAP compositions of the present invention provide may be used to increase natural saprophytic or added beneficial microorganisms, to reduce population density of natural microorganisms by inhibitory activity of secondary metabolites produced by added beneficial microorganisms or by antimicrobial agents that are selectively added in the SAP compositions. Natural saprophytic or added beneficial microorganisms that are increased in population by using the SAP composition of the present invention may compete more effectively with plant pathogenic microorganisms for microbial nutrients and spaces that result in reduced disease development. When the SAP compositions added with certain microorganisms that have insecticidal activity, the insecticidal microorganisms may have higher chance of suppressing harmful insects on surfaces when applied properly to plant surfaces. When the SAP compositions added with herbicidal microorganisms, the herbicidal microorganisms may have higher chances of suppressing weedy plants in selective ways when applied properly to surfaces of weedy plants. When the SAP compositions added with specific beneficial microorganisms that are inhibitory to growth of snails, cockroaches or ants, the beneficial microorganisms may also have higher chances of suppressing or eliminating harmful animals when applied properly to animal surfaces.

### BEST MODE FOR CARRYING OUT THE INVENTION

To achieve the above-described purpose of the invention, the present invention provides compositions comprising SAP to increase microbial population or biological activities of beneficial microorganisms on plant, soil or animal surfaces of plant, soil and animal.

The SAP composition of the present invention additionally may contain at least one nutrient that can be used by natural saprophytic or added beneficial microorganisms.

The SAP composition of the present invention additionally may contain at least one beneficial microorganism that can inhibit plant pathogens, harmful insects or weedy plants or that can promote plant growth.

The SAP composition of the present invention additionally may contain at least one beneficial microorganism that can inhibit harmful animals.

The SAP composition of the present invention additionally may contain at least one surfactant to achieve even spreading of the composition on surfaces.

The SAP composition of the present invention additionally may contain at least one single or multiple mineral salt to improve wettability of the composition in water.

The SAP composition of the present invention is characterized in that SAP is starch-based SAP.

The SAP composition of the present invention is characterized in that the SAP content ranges from 20% to 95% by weight.

The SAP composition of the present invention is characterized in that the SAP content ranges from 0.02% to 1.0% by weight in a given water volume, when the composition is diluted with water.

The SAP composition of the present invention is characterized in that the surfaces are surfaces of plant leaves, stems, branches, flowers or fruits, surfaces of ground soil, or surfaces of animals.

The SAP composition of the present invention is characterized in that the microbial nutrient is PLNT powder of culture broth that consists of potato dextrose broth, Luria broth base, nutrient broth and tryptic soy broth each comprised in an equal amount by weight.

The SAP composition of the present invention is characterized in that the plants are selected from cucumber, red pepper, potato, rice, tomato, barley, wheat, pear or rose.

The SAP composition of the present invention is characterized in that the beneficial microorganisms are selected from bacterial genera of Bacillus, Paenibacillus or Streptomyces, fungal genera of Trichoderma, Ampelomyces or Acremonium.

The SAP composition of the present invention is characterized in that the plant pathogens are selected from Magnaporthe grisea, Thanatephorus cucumeris, Phytophthora capsici, Botrytis cinerea, Puccinia graminis, Blumeria graminis=Erysiphe graminis or Sphaerotheca fusca.

The SAP composition of the present invention is characterized in that the harmful insect is genus Plutella.

The SAP composition of the present invention is characterized in that the weedy plants are the plants that are growing in field in a given time and space that are not intended by plant growers.

The SAP composition of the present invention is characterized in that the harmful animals are cockroaches, ants or snails.

The present invention provides use of the SAP compositions on plant surfaces or soil surfaces.

The present invention provides use of the SAP compositions to suppress harmful organisms on surfaces or to promote plant growth.

Hereafter, more specific descriptions of the present invention are given.

The present invention is about compositions of increasing biological activities or population density of natural saprophytic or added beneficial microorganisms on surfaces and their uses. The compositions of the present invention comprise SAP that is super absorbent polymer of water. Depending on application and necessity, the compositions of the present invention may contain microbial nutrients or beneficial microorganisms. Furthermore, wetting agents or surfactants may be added to improve wettability in water and spreadability on surfaces, respectively. In the present invention, a key material used to increase water amount and to extend supply time of water available for microorganisms are super absorbent polymer (hereafter referred as “SAP”). Microbial nutrients were composed of culture broth that is widely used by microbiologists in laboratory for research. Beneficial microorganisms were obtained from a commercial provider or obtained from researchers who study the corresponding microorganisms. In the compositions of the present invention, mineral salts and surfactants are selected and properly used to improve wettability in water, and spreadability of the composition on surfaces, respectively.

### 1. SAP (Super Absorbent Polymer)

The SAP used mainly in the present invention is starch-based, specifically Zeba (product brand name), comprising a major component starch-g-poly(2-propenamide-co-2-propenoic acid) potassium salt (CAS Registry No. 107830-79-5) that is manufactured by Absorbent Technologies, Inc. The SAP content was 50% of the compositions by weight prior to dilution in water, and 0.02%-1.0% by weight in a given water volume when diluted in water. The SAP content in said SAP compositions and examples, the SAP content was 0.5% by weight in a given water volume. For comparison purpose, an acrylamide-based SAP (GS-3000), manufactured by Kolon Industries Inc., was used instead of Zeba, which resulted in similar effects of increasing microbial population when tested on cucumber leaves. In the present invention, SAP was milled into fine powders to have a diameter the same or less than 0.1 mm. Smaller the SAP particles, better the SAP suspension after dilution spreads over wide range of plant surfaces. However, the above-described kinds, contents and diameters of SAP were given to exemplify the present invention and does not restrict kinds and a method of preparing SAP that are comprised in compositions of the present invention.

### 2. Microbial Nutrients

In the present invention, a microbial nutrient, named as PLNT powder medium, was used. The PLNT powder medium consists of potato dextrose broth, Luria broth base, nutrient broth and tryptic soy broth that are all manufactured by Difco Laboratories, USA. One quarter of recommended use of each medium was combined to prepare the PLNT powder medium. Good growth of various microorganisms was confirmed on PLNT agar medium. The PLNT powder medium was used with 5% by weight in the composition of the present invention. However, the PLNT powder broth was used as an example of the present invention and kinds or contents of microbial nutrients are not limited thereto. Rather, other microbial nutrients that provide carbon, nitrogen, amino acids, minerals or vitamins required for microbial growth can be also used.

### 3. Beneficial Microorganisms

All beneficial microorganisms used in the present invention were obtained from a commercial provider or from researchers. Beneficial microorganisms used for plant protection from plant pathogens belong to bacterial genera of Bacillus, Paenibacillus or Streptomyces or fungal genera of Trichoderma, Ampelomyces or Acremonium. Beneficial microorganism used for plant protection from harmful insect belongs to bacterial genus of Bacillus thuringiensis, and that used for plant protection from weedy plant belongs to bacterial genus of Botrytis cinerea. Beneficial microorganism used for growth promotion of plants belongs to bacterial genus of Bacillus vallismortis. References of each beneficial microorganism are given below.

### <Reference for Beneficial Microorganisms>

1. Beneficial Microorganisms Used for Plant Protection from Plant Pathogens
(1) Paenibacillus elgii (SD17)
A. Kim, D. S., Bae, C. Y., Jeon, J. J., Chun, S. J., Oh, H. W., Hong, S. G., Baek, K. S., Moon, E. Y., and Bae, K. S. 2004. Paenibacillus elgii sp. nov., a novel species with broad antimicrobial activity. International Journal of Systemic and Evolutionary Microbiology 54:2031-2035.
B. Kim, D. S., Bae, C. Y., Kim, D. H, Chun, S. J., Choi, S. W., and Choi, K. H. 2005. Paenibacillus elgii SD17 as a biocontrol agent against soil-borne turf diseases. Plant Pathology Journal 21:328-333.
C. Korean Patent No. 0457025
   (2) Bacillus subtilis (QST713)
A. U.S. Pat. No. 6,060,051
   (3) Bacillus subtilis (GB-0365)
A. Korean Patent No. 294023

### (4) Streptomyces sp. (A020645)

A. Lee, H. B., Cho, J. W., Park, D. J., Li, C. T., Ko, Y. H., Song, J. H., Koh, J. S., Kim, B. J. Kim, C. J. 2004. In vivo screening for biocontrol agents (BCAs) against Streptomyces scabiei causing potato common scab. Plant Pathology Journal 20: 110-114.
B. Korean Patent No. 0563298
   (5) Trichoderma harzianum (YC459)
A. Lee, S. K., Sohn, H. B., Kim, G. G. and Chung, Y. R. 2006. Enhancement of biologic al control of Botrytis cinerea on cucumber by foliar sprays and bed potting mixes of Trichoderma harzianum YC459 and its application on tomato in the greenhouse. Plant Pathology Journal 22:283-288.
B. Korean Patent No. 0417632
   (6) Ampelomyces quisqualis (AQ94013)
B. Korean Patent No. 0332480
   (7) Acremonium strictum (BCP)
A. Kim, J. C., Choi, G. J., Kim, H. J., Kim, H. T., Ahn, J. W. and Cho, K. Y. 2002. Verlamelin, an antifungal compound produced by a mycoparasite, Acremonium strictum. Plant Pathology Journal 18:102-105.
B. Korean Patent No. 0299768
   2. Beneficial Microorganisms Used for Plant Protection from Harmful Insects
   (8) Bacillus thuringiensis subsp. aizawai (NT0423)
A. Korean Patent No. 0280380

### 3. Beneficial Microorganisms Used for Plant Growth Promotion

(9) Bacillus vallismortis (EXTN-1)
A. Park, K. S., Paul, D. and Yeh, W. H. 2006a. Bacillus vallismortis EXTN-1-mediated growth promotion and disease suppression in rice (Oryza sativa L.). Plant Pathology Journal 22:278-282.
B. Park, K. S., Paul, D., Ryu, K. R., Kim, E. Y, and Kim, Y. K. 2006b. Bacillus vallismortis strain EXTN-1 mediated systemic resistance against Potato Virus X and Y (PVX & PVY) in the field. Plant Pathology Journal 22:360-363.
C. Park, K. S., Paul, D., Kim, Y. K., Nam, K. W., Lee, Y. K., Choi, H. W. and Sang Y. L. 2007. Induced systemic resistance by Bacillus vallismortis EXTN-1 suppressed bacterial wilt in tomato caused by Ralstonia solanacearum. Plant Pathology Journal 23:22-25.
D. Korean Patent No. 0379022

In the present invention, for experimental purpose, microorganisms obtained from one liter of water shaken vigorously in a 250 ml of Erlenmeyer flask with 100 g of fresh cucumber leaves sampled from adult cucumber plants, left statically for 30 minutes, shaken vigorously again, and then filtered through two layers of sterile cheese cloths (hereafter referred as “leaf wash water”) were used as natural microorganisms. In the present invention, plant pathogens were artificially cultured. For plant pathogens that are not culturable due to their intrinsic obligate parasitism, inoculum was obtained from plants which are infected with the pathogen and show typical symptom of the infection. In the present invention, larva of Plutella species was used as a harmful insect. In principle, most bacteria or fungi listed on “The manual of biocontrol agent” published by BCPC in 2004 can be considered as beneficial microorganisms. In the manual, beneficial microorganisms that are established on plant surface or soil surface in advance to arrival of plant pathogens compete with plant pathogens for nutrition or space that results in plant protection. In case of plant protection from harmful insects, beneficial microorganisms are taken into inside harmful insects and results in death or inactivation of harmful insects. In case of plant protection from weedy plants, beneficial microorganisms cause diseases that are pathogenic specifically only to the weedy plants. Therefore, the experiments in the present invention are only to illustrate the present invention and do not limit kinds of beneficial microorganisms or harmful organisms.

### 4. Preparation of Microbial Compositions

Microbial compositions in the present invention was prepared by milling and mixing of each component as powder with particle size that is the same or less than 0.1 mm in average. Among the components of the present invention, SAP was the starch-based SAP. Microbial nutrient was the PLNT. Beneficial microorganisms were obtained from a commercial provider or obtained, isolated for pure culture in laboratory, cultured and prepared as powder by a freeze-drying technique. In addition, 5% of surfactant in powder was used to accomplish even spreading of the composition on plant surface. In the present invention, the surfactant was softanol (CAS Registry No. 68131-40-8) manufactured by Ineos Oxide Ltd., UK. Sofanol was adsorbed to white carbon supplied by Jungwoo Chemical, Korea, with 1:1 ratio by weight. More specifically, softanol is a non-ionic surfactant obtained by adding ethylene oxide to linear secondary alcohol with 12-14 alkyl carbons. Complex mineral salts were used in the microbial composition to improve wettability of the composition in water. Mirigun powder, manufactured by Daeyoo, Korea, was added as a wetting agent with 30% ratio by weight to microbial compositions. Mirigun is a fertilizer to provide minerals required for plant growth. In the present invention, Mirigun was used as carrier and to improve wettability in water based on its high solubility in water and high specific gravity. A single mineral salt such as calcium chloride can be also applied to microbial compositions in the present invention. For compositions without containing beneficial microorganisms, Mirigun powder was used with 40% ratio by weight. Content of each component in 100 g composition was described in Table 1.

TABLE 1
Composition

| | | | Beneficial microorganisms | | | | |
|---|---|---|---|---|---|---|---|
| | | Microbial | | | Population Density of | | |
| Composition | SAP (g) | nutrients (g) | Code | g | microorganism (log cfu/g) | Surfactant (g) | Carrier (g) |
| Composition 1 | 50 | 0 | — | 0 | 0 | 5 | 40 |
| Composition 2 | 50 | 5 | — | 0 | 0 | 5 | 40 |
| Composition 3 | 50 | 5 | SD17 | 10 | 8.0 | 5 | 30 |
| Composition 4 | 50 | 5 | QST713 | 10 | 9.7 | 5 | 30 |
| Composition 5 | 50 | 5 | GB-0365 | 10 | 7.5 | 5 | 30 |
| Composition 6 | 50 | 5 | A020645 | 10 | 8.3 | 5 | 30 |
| Composition 7 | 50 | 5 | YC459 | 10 | 8.0 | 5 | 30 |
| Composition 8 | 50 | 5 | AQ94013 | 10 | 7.0 | 5 | 30 |
| Composition 9 | 50 | 5 | BCP | 10 | 7.0 | 5 | 30 |
| Composition 10 | 50 | 5 | NT0423 | 10 | 7.0 | 5 | 30 |
| Composition 11 | 50 | 5 | EXTN-1 | 10 | 7.7 | 5 | 30 |
| Beneficial microorganism 1 | 0 | 0 | SD17 | 10 | 8.0 | 5 | 30 |
| Beneficial microorganism 2 | 0 | 0 | QST713 | 10 | 9.7 | 5 | 30 |
| Beneficial microorganism 3 | 0 | 0 | GB-0365 | 10 | 7.5 | 5 | 30 |
| Beneficial microorganism 4 | 0 | 0 | A020645 | 10 | 8.3 | 5 | 30 |
| Beneficial microorganism 5 | 0 | 0 | YC459 | 10 | 8.0 | 5 | 30 |
| Beneficial microorganism 6 | 0 | 0 | AQ94013 | 10 | 7.0 | 5 | 30 |
| Beneficial microorganism 7 | 0 | 0 | BCP | 10 | 7.0 | 5 | 30 |
| Beneficial microorganism 8 | 0 | 0 | NT0423 | 10 | 7.0 | 5 | 30 |
| Beneficial microorganism 9 | 0 | 0 | EXTN-1 | 10 | 7.7 | 5 | 30 |

^{A)}Population density of microorganism was expressed as logarithmic value of the colony forming unit per gram powder (i.e., log cfu).
^{B)}Surfactant: Powder of softanol adsorbed to white carbon with 1:1 ratio by weight.
^{C)}Carrier: Mirigun powder

These compositions are only examples and do not limit kinds of SAP, beneficial microorganisms, microbial nutrients, surfactants or carriers that can be used for the compositions of the present invention.

The present invention will now be described in greater detail with reference to the following examples. However, it is only to specifically exemplify the present invention and in no case the scope of the present invention is limited by these examples.

### Example 1

### Dynamics of Microbial Populations

In this Example, effect of each composition on population density of natural or added beneficial microorganisms present on plant surface was monitored with time. For experimental purpose, natural microorganisms were defined as microorganisms obtained from one liter of water shaken vigorously in a 250 ml of Erlenmeyer flask with 100 g of fresh cucumber leaves sampled from adult cucumber plants, maintained undisturbed for 30 minutes, shaken vigorously again, and then filtered through two layers of sterile cheese cloths (hereafter referred as “leaf wash water”). When cultured on LB agar medium, approximately 2×10⁵ cfu/ml (=5.3 log cfu/ml) of natural microorganisms were detected. Of these, bacteria were present in a dominant amount of 90% of the total population. Each composition from the present invention at 0.5% by weight was added to the leaf wash water to become 10 ml in total volume. Seven discs of cucumber leaf in 3 cm diameter were soaked for 30 min and placed on a Petri dish at room temperature. In addition, for comparison purpose, each beneficial microorganism was added to 10 ml of the leaf wash water at the same rate and prepared as above. Composition without microbial nutrient or beneficial microorganism was also prepared for comparison purpose. Each preparation was incubated for 0, 1, 2, 3, 5 and 7 day after treatment on LB agar media at room temperature. Microbial population was properly counted 3-7 days after incubation by dilution plating method. Beneficial microorganisms were differentiated by their unique colony form on the media, and all the others were considered as natural microorganisms. These results are summarized in Table 2.

TABLE 2
Population dynamics of beneficial or natural microorganisms on discs of cucumber leaf

| | Microbial population density (log cfu/leaf disc)^{A)} | | | | | | | | | | | |
|---|---|---|---|---|---|---|---|---|---|---|---|---|
| | 0 DAT | | 1 DAT | | 2 DAT | | 3 DAT | | 5 DAT | | 7 DAT | |
| Treatment | NM^{B)} | BM^{C)} | NM | BM | NM | BM | NM | BM | NM | BM | NM | BM |
| Composition 1 | 4.1 | 0.0 | 4.5 | 0.0 | 5.6 | 0.0 | 5.3 | 0.0 | 5.1 | 0.0 | 5.1 | 0.0 |
| Composition 2 | 4.3 | 0.0 | 5.3 | 0.0 | 6.5 | 0.0 | 6.3 | 0.0 | 6.0 | 0.0 | 5.7 | 0.0 |
| Composition 3 | 4.1 | 5.1 | 5.1 | 5.7 | 4.1 | 6.5 | 3.8 | 6.1 | 3.5 | 6.0 | 3.1 | 5.9 |
| Composition 4 | 4.0 | 7.0 | 4.5 | 7.3 | 4.0 | 7.8 | 3.5 | 8.0 | 3.5 | 7.5 | 3.5 | 7.0 |
| Composition 5 | 4.1 | 4.3 | 5.3 | 5.0 | 5.5 | 5.8 | 5.3 | 6.3 | 4.8 | 6.0 | 5.0 | 6.0 |
| Composition 6 | 4.0 | 5.1 | 5.1 | 5.0 | 4.7 | 5.7 | 4.5 | 6.0 | 4.1 | 5.5 | 3.7 | 6.0 |
| Composition 7 | 4.2 | 4.8 | 5.0 | 5.5 | 5.5 | 5.8 | 6.0 | 5.5 | 6.0 | 5.3 | 6.0 | 5.1 |
| Composition 8 | 4.1 | 4.1 | 5.3 | 4.6 | 4.9 | 5.2 | 5.1 | 5.5 | 4.9 | 6.0 | 4.9 | 5.5 |
| Composition 9 | 4.2 | 4.0 | 5.4 | 4.3 | 5.9 | 5.1 | 5.4 | 5.5 | 5.4 | 5.2 | 5.0 | 5.0 |
| Composition 10 | 4.0 | 7.2 | 5.5 | 7.7 | 6.1 | 7.8 | 6.5 | 7.3 | 6.3 | 7.1 | 6.2 | 6.9 |
| Composition 11 | 3.9 | 4.5 | 4.5 | 5.0 | 5.6 | 6.0 | 6.1 | 5.5 | 6.0 | 5.1 | 6.2 | 5.0 |
| Beneficial microorganism 1 | 4.0 | 5.2 | 5.0 | 5.5 | 5.1 | 5.5 | 5.0 | 5.1 | 4.5 | 5.0 | 4.2 | 4.8 |
| Beneficial microorganism 2 | 3.8 | 6.8 | 4.8 | 7.0 | 5.0 | 6.8 | 4.5 | 6.5 | 4.0 | 6.0 | 3.8 | 5.5 |
| Beneficial microorganism 3 | 4.2 | 4.5 | 5.1 | 5.0 | 5.2 | 5.5 | 4.8 | 5.1 | 4.0 | 4.8 | 3.8 | 4.5 |
| Beneficial microorganism 4 | 4.3 | 5.0 | 5.2 | 5.0 | 5.3 | 5.1 | 4.6 | 5.1 | 4.1 | 4.5 | 4.0 | 4.0 |
| Beneficial microorganism 5 | 4.0 | 5.0 | 5.0 | 5.1 | 5.1 | 5.1 | 4.5 | 5.3 | 3.8 | 4.8 | 3.5 | 4.5 |
| Beneficial microorganism 6 | 3.8 | 4.3 | 4.8 | 4.6 | 4.5 | 4.8 | 3.8 | 4.8 | 3.5 | 4.0 | 3.5 | 3.8 |
| Beneficial microorganism 7 | 4.1 | 4.1 | 5.0 | 4.5 | 5.1 | 4.8 | 4.8 | 4.0 | 4.0 | 3.8 | 3.8 | 4.0 |
| Beneficial microorganism 8 | 4.0 | 7.0 | 5.0 | 7.5 | 5.0 | 7.1 | 4.5 | 6.5 | 3.8 | 6.0 | 3.5 | 6.1 |
| Beneficial microorganism 9 | 3.7 | 4.2 | 4.5 | 4.8 | 4.7 | 4.5 | 4.5 | 4.1 | 3.5 | 3.8 | 3.1 | 4.0 |
| Control(WLW)^{D)} | 4.0 | 0.0 | 4.2 | 0.0 | 3.9 | 0.0 | 4.0 | 0.0 | 3.9 | 0.0 | 3.7 | 0.0 |

^{A)}Mcirobial population density was counted on basis of colony forming unit and transformed into logarithmic number.
^{B)}NM = Natural microorganisms
^{C)}BM = Beneficial microorganisms that were artificially added to the leaf wash water
^{D)}WLR = Leaf wash water

As shown in Table 2, most of the beneficial or natural microorganisms were increased in their population when properly used with the compositions of the present invention. Population density of natural microorganism was increased up to 100-fold at certain time point for some compositions. However, in case with composition 3 containing beneficial microorganism 1 and composition 4 containing beneficial microorganism 2, the density of the natural microorganisms was decreased or their increase rate was reduced. This observation may be explained by antibiosis of the beneficial microorganisms 3 and 4 that have antimicrobial activity by producing secondary metabolites. For example, Paenibacillus elgii SD17 was reported with its broad antimicrobial activity (Kim, D. S., Bae, C. Y., Kim, D. H, Chun, S. J., Choi, S. W., and Choi, K. H. 2005. Paenibacillus elgii SD17 as a biocontrol agent against soil-borne turf diseases. Plant Pathology Journal 21:328-333). Based on results in Table 1, it was found that compositions of the present invention provides more water and microbial nutrients to microbial population on cucumber leaf and results in increased population density of natural or beneficial microorganisms. In addition, in case with beneficial microorganisms with antimicrobial activity, the beneficial microorganisms were well established by decreased population density of natural microorganisms or by reduced increase of population density of natural microorganisms in accordance with the application of the composition of the present invention.

### Example 2

### Plant Protection Activity of Compositions on Plant Seedlings from Plant Pathogens

In this Example, compositions of the present invention were tested in growth chamber for their plant protection activity against plant pathogens; Magnaporthe grisea, Thanatephorus cucumeris, Phytophthora capsici, Botrytis cinerea, Puccinia graminis and Blumeria graminis (i.e., Erysiphe graminis). The experiments were conducted on basis of general screening methods of chemical fungicides (Choi, G. J, Kim, J.-C., Lee, S.-W., Jang, K. S., Kim, J.-S., and Cho, K. Y. 2002. Antifungal activities of several plant extracts against wheat leaf rust. The Korean Journal of Pesticide Science 6(2): 87-95. In Korean). One main difference was that each plant pathogen was inoculated 3 days after treatment of the plant with each composition with 200-fold dilution by spraying on plant surfaces. Treated plants were maintained by general practice, and plant protection activity was estimated by control value. Control value (CV) is estimated by the following formula; CV(%)={1−(diseased area ratio of treatment group/diseased area ratio of non-treatment group)}×100.

TABLE 3
Protection activity of each composition and beneficial
microorganism against plant diseases

| | Control value (%) | | | | | |
|---|---|---|---|---|---|---|
| Treatment | RLB^{A)} | RSB^{B)} | TGM^{C)} | TLB^{D)} | WLR^{E)} | BPM^{F)} |
| Composition 1 | 24 | 20 | 17 | 27 | 4 | 30 |
| Composition 2 | 27 | 25 | 30 | 37 | 7 | 45 |
| Composition 3 | 30 | 25 | 50 | 84 | 20 | 25 |
| Composition 4 | 11 | 15 | 45 | 79 | 80 | 28 |
| Composition 5 | 45 | 55 | 65 | 47 | 15 | 34 |
| Composition 6 | 30 | 45 | 55 | 45 | 40 | 75 |
| Composition 7 | 25 | 65 | 75 | 55 | 25 | 25 |
| Composition 8 | 35 | 30 | 30 | 40 | 45 | 70 |
| Composition 9 | 25 | 35 | 40 | 25 | 25 | 30 |
| Composition 10 | 30 | 20 | 25 | 25 | 20 | 25 |
| Composition 11 | 45 | 30 | 30 | 35 | 65 | 70 |
| Beneficial microorganism 1 | 28 | 0 | 35 | 58 | 0 | 0 |
| Beneficial microorganism 2 | 0 | 5 | 37 | 73 | 83 | 8 |
| Beneficial microorganism 3 | 25 | 35 | 37 | 27 | 0 | 7 |
| Beneficial microorganism 4 | 10 | 0 | 5 | 0 | 35 | 55 |
| Beneficial microorganism 5 | 0 | 45 | 50 | 35 | 0 | 0 |
| Beneficial microorganism 6 | 0 | 5 | 0 | 15 | 25 | 55 |
| Beneficial microorganism 7 | 5 | 10 | 20 | 0 | 5 | 5 |
| Beneficial microorganism 8 | 5 | 0 | 0 | 0 | 5 | 0 |
| Beneficial microorganism 9 | 25 | 0 | 5 | 5 | 25 | 35 |

RCB^{A)}Rich blast caused by Maganaporthe grisea
RSB^{B)}Rich sheath blight caused by Thanatephorus cucumeris
TGM^{C)}Tomato gray mold caused by Botrytis cinerea
TLB^{D)}Tomato late blight caused by Phytophthora infestans
WLR^{E)}Wheat leaf rust caused by Puccinia recondita
BPM^{F)}Barley powdery mildew caused by Blumeria graminis f. sp. Hordei

In Table 3, SAP compositions of the present invention in general showed protection activity against the plant diseases that resulted in increased control value. More specifically, overall increase in control value by the composition 1 was observed for all the diseases. Overall increase of control value by the composition 2 was also observed for all the diseases. The composition 2 resulted in somewhat higher control values than the composition 1. Overall increase of control value was also observed with the composition 3 to composition 11. Especially, control value was improved for the compositions that showed protection activity against plant diseases only with each of beneficial microorganisms. The beneficial microorganism 1 by itself showed protection activity against tomato late blight (TLB) and tomato gray mold (TGM) whereas the composition 3 containing the beneficial microorganism 1 showed improved protection activity against the same diseases and additionally showed a certain protection activity against rice blight (RCB), rice sheath blight (RSB), wheat leaf rust (WLR) and barley powdery mildew (BPM). The beneficial microorganism 2 by itself showed protection activity against TLB and BPM, whereas the composition 4 containing the beneficial microorganism 2 showed improved protection activity against TLB and BPM and additionally showed a certain protection activity against other diseases. The beneficial microorganism 3 by itself showed protection activity against RCB, RSB, TGM and TLB, whereas the composition 5 containing the beneficial microorganism 3 showed improved protection activity against RCB, RSB, TGM and TLB and additionally showed a certain protection activity against other diseases. The beneficial microorganism 4 by itself showed protection activity against WLR and BPM, whereas the composition 6 containing the beneficial microorganism 4 showed improved protection activity against WLR and BPM and additionally showed a certain protection activity against other diseases. The beneficial microorganism 5 by itself showed protection activity against TGM and RSB, whereas the composition 7 containing the beneficial microorganism 5 showed improved protection activity against TGM and RSB and additionally showed a certain protection activity against other diseases. The beneficial microorganism 6 by itself showed protection activity against BPM and WLR, whereas the composition 8 containing the beneficial microorganism 6 showed improved protection activity against BPM and WLR and additionally showed a certain protection activity against other diseases. The beneficial microorganism 7 by itself showed protection activity against TLB and RSB, whereas the composition 9 containing the beneficial microorganism 7 showed improved protection activity against TLB and RSB and additionally showed a certain protection activity against other diseases. The beneficial microorganism 8 that has protection activity against insect larva of Plutella species did not show any protection activity against the plant diseases, whereas the composition 10 showed containing the beneficial microorganism 8 showed a certain protect activity similar to the composition 2 against the plant diseases. The beneficial microorganism 9 that induces disease resistance by itself showed protection activity against BPM and WLR, whereas the composition 11 containing the beneficial microorganism 9 showed improved protection activity against BPM and WLR and additionally showed a certain protection activity against other diseases. As described above, each of the compositions of the present invention in general showed higher control value than each of the microorganisms. The composition 1 or composition 2 not comprising the beneficial microorganisms also showed a certain level of increased control value.

However, considering the Example 2 that was conducted in a system which is usually employed for the evaluation of chemical pesticides for short periods in growth chamber that was established with stable conditions including temperature, humidity and lighting for disease development or insect growth, there was a certain limitation to confirm protection activity. Thus, in order to confirm protection activity of the compositions of the present invention against plant disease, natural condition is considered more desirable because of plant surfaces to be exposed to fluctuating moisture, temperature and lighting in day and night. Plants can be also grown for longer period. Therefore, for better confirmation of the effect of the present invention, by using adult plants under general conditions for cultivation or similar conditions, an experiment was further carried out repeatedly to confirm the efficacy of the composition of the present invention.

### Example 3

### Protection Activity Against Cucumber Powdery Mildew and Population Dynamics of Microbial Population

Of the compositions of the present invention, some compositions were confirmed for their protection activity against cucumber powdery mildew caused by Sphaerotheca fusca that naturally occur on adult plants in a green house. Cucumber seedlings grown for 1 month after seeding were transplanted into pots (15 cm in diameter, 20 cm in depth) that were filled with a fertile soil (Bunong Sangto No. 5) manufactured by Biomedia, Korea, and additionally grown until cucumber plants grew up to the fifth or sixth leaf stages by general practice in the green house. Then, water suspension of the compositions 1, 2, 3 and 4, the beneficial microorganisms 1 and 2, and a chemical fungicide with active ingredient azoxystrobin were respectively sprayed on cucumber surfaces and leaves with 7 days of application interval for comparative studies. The experiments were prepared by randomized complete block design with three replications. Four cucumber plants were included for each replication. Spray suspension comprises each composition of the present invention or the beneficial microorganisms that are 200-fold diluted with water. Each beneficial microorganism was prepared to have similar number of beneficial microorganisms when diluted with the same amount of water. Spray suspension was sufficiently applied on cucumber leaves and surfaces by hand-held sprayer until the suspension drops off plants. Cucumber powdery mildew was induced to occur naturally on a few cucumber plants in the green house. In fact, the occurrence of the disease was observed when cucumbers reached the fifth or sixth leaf stage. Protection activity was estimated on the day of each application according to a guideline of Rural Development Administration, Korea. Additional estimations were made on the seventh and fourteenth days after treatment 3 (7 DAT 3 and 14 DAT 3). The protection activities against cucumber powdery mildew were given in Table 4.

TABLE 4
Protection activity against cucumber powdery mildew

| | Control value (%) | | | | |
|---|---|---|---|---|---|
| Treatment | 0 DAT1 | 0 DAT2 | 0 DAT3 | 7 DAT3 | 14 DAT3 |
| Composition 1 | 0 | 25 b^{B)} | 32 c | 35 c | 27 cd |
| Composition 2 | 0 | 32 b | 43 bc | 43 c | 35 c |
| Composition 3 | 0 | 58 a | 77 a | 80 a | 76 a |
| Composition 4 | 0 | 55 a | 62 ab | 65 ab | 65 ab |
| Beneficial microorganism 1 | 0 | 25 b | 38 c | 40 c | 45 c |
| Beneficial microorganism 2 | 0 | 43 ab | 30 c | 37 c | 38 c |
| Chemical fungicide^{A)} | 0 | 27 b | 32 c | 15 d | 12 d |

^{A)}Chemical fungicide with azoxystrobin as active ingredient
^{B)}The same letters in each column are not significantly different by Duncan's multiple range test at P = 0.05.

As shown in Table 4, the compositions 1 and 2 resulted in a certain level of protection activity. The compositions 3 and 4 showed improved protection activities when compared with each beneficial microorganism. The chemical fungicide, azoxystrobin, did not show good protection activity for this test. This was considered due to resistance of pathogen to fungicide azoxystrobin.

In parallel, microbial populations on cucumber leaves were also monitored. On the same day prior to each application using a cork borer, three leaf discs (3 cm in diameter) were collected from the third main leaf of cucumber plant for each treatment. The collected leaf discs were put into 10 ml of sterile water for 30 minutes and shaken hard by hand to prepare the leaf wash water. Microbial population was measured by 10-fold serial dilution plating method or using LB agar medium. The beneficial microorganisms were counted on the basis of their own unique colony forms on the LB agar media. The result is given in Table 5.

TABLE 5
Population dynamics of microorganisms on cucumber leaf surface

| | Population density (log cfu/leaf disc)^{A)} | | | | | | | | | |
|---|---|---|---|---|---|---|---|---|---|---|
| | 0 DAT 1 | | 0 DAT2 | | 0 DAT3 | | 7 DAT3 | | 14 DAT3 | |
| Treatment | NM^{B)} | BM^{C)} | NM | BM | NM | BM | NM | BM | NM | BM |
| Composition 1 | 4.0 | 0.0 | 4.5 | 0.0 | 5.6 | 0.0 | 5.3 | 0.0 | 5.0 | 0.0 |
| Composition 2 | 4.3 | 0.0 | 5.3 | 0.0 | 6.9 | 0.0 | 7.3 | 0.0 | 6.0 | 0.0 |
| Composition 3 | 4.0 | 5.0 | 4.5 | 6.5 | 3.8 | 7.0 | 3.5 | 7.0 | 3.0 | 6.3 |
| Composition 4 | 4.0 | 7.0 | 4.0 | 7.3 | 4.0 | 7.8 | 3.3 | 8.0 | 3.0 | 7.5 |
| Beneficial microorganism 1 | 4.0 | 5.0 | 4.8 | 5.5 | 4.5 | 6.0 | 4.3 | 5.3 | 4.3 | 5.0 |
| Beneficial microorganism 2 | 4.0 | 7.0 | 4.5 | 6.5 | 4.0 | 6.7 | 4.0 | 6.0 | 4.0 | 5.7 |
| Chemical fungicide | 4.3 | 0.0 | 5.0 | 0.0 | 4.0 | 0.0 | 4.3 | 0.0 | 4.0 | 0.0 |
| Non-treatment | 4.0 | 0.0 | 4.0 | 0.0 | 4.3 | 0.0 | 4.0 | 0.0 | 4.5 | 0.0 |

^{A)}Mcirobial population density was counted on basis of colony forming unit and transformed into logarithmic number.
^{B)}NM = Natural microorganisms
^{C)}BM = Beneficial microorganisms

In Table 5, microbial population density when treated with the compositions was significantly increased when compared to the treatments only with each beneficial microorganism. Especially, for the composition 2, population density of natural microorganisms was increased 100 times, when compared with the non-treatment. Also microbial population of the composition 2 was in overall higher than that of the composition 1. Further, the composition 3 containing the beneficial microorganism 1 and the composition 4 containing the beneficial microorganism 2 showed a certain activity to reduce population density of natural microorganisms. This trend indicates that the protection activities in Table 4 were due to increased population density of each beneficial microorganism. This experiment also confirms that population dynamics of microorganisms shown in Table 5 was similarly reproduced with adult cucumber plants in the green house.

### Example 4

### Protection Activity from Harmful Insects

Composition 10 of the present invention was tested in a laboratory to evaluate insecticidal activity to diamondback moth (Plutella xylostella) that harms plants belonging to Cruciferae. Cabbage seedlings were grown in a green house, and their leaf discs (5 cm in diameter) were sampled and soaked for 30 seconds in suspension of the composition 10 that was 2000-fold diluted. Those leaf discs were taken out and dried in shade in a hood. Ten larvae of Plutella xylostella that had been grown up to the third stage larvae were placed on the leaf discs and assessed for insecticidal activity at 24, 48, 72, 96 and 120 hours after the placement. The experiment was prepared by randomized block design with three replications. The insecticidal activity was assessed according to a guideline of Rural Development Administration (Korea). The result is given in Table 6.

TABLE 6
Insecticidal activity against diamondback moth (Plutella xylostella)

| | Insecticidal activity (%)^{A)} | | | | |
|---|---|---|---|---|---|
| Treatment | 24 hour^{B)} | 48 hour | 72 hour | 96 hour | 120 hour |
| Composition 10 | 30 ab^{C)} | 80 a | 100 a | 100 a | 100 a |
| Beneficial microorganism 8 | 40 a | 70 ab | 80 b | 90 ab | 100 a |

^{A)}Insecticidal activity (%) is percentage of dead larvae at each time.
^{B)}Hours after placement on the leaf discs
^{C)}The same letters in each column are not significantly different by Duncan's multiple range test at P = 0.05.

As shown in Table 6, the composition 10 of the present invention resulted in 100% of insecticidal activity at 72 hour after the treatment. The insecticidal activity was significantly higher than the case in which the beneficial microorganism 8 was used alone. Furthermore, time to reach 100% of insecticidal activity was shorter for the composition 10 than for the beneficial microorganism 8.

This example indicates that the compositions of the present invention can be applied not only for the plant protection from plant pathogens but also for the plant protection from harmful insects. In a similar approach, the compositions of the present invention are considered to be useful for extended use by increasing population of beneficial microorganisms to suppress harmful organisms such as cockroaches, ants, snails and so on.

### Example 5

### Herbicidal Activity

When some plant pathogens specifically infect limited number of weedy plants, compositions of the present invention that contain these plant pathogens can be used to suppress or control weedy plants. In this example, tomato was taken as an example of weedy plants. The compositions 1 and 2 of the present invention that contain spores of Botrytis cinerea at 8.00 log cfu/ml were respectively diluted at 200-fold rate and sufficiently sprayed with 50 ml for each tomato seedling until dropping off. Herbicidal activity was estimated with a similar method used in the Example 2. Herbicidal activity was estimated at 3 and 7 days after inoculation (3 DAI and 7 DAI) based on percentage of infected leaf area. The result is given in Table 7.

TABLE 7
Herbicidal activity against tomato seedlings

Infect leaf area (%)

| Treatment | 3 DAI | 7 DAI |
|---|---|---|
| Botrytis cinerea only | 25 b | 35 c |
| Composition 1 + | 38 b | 58 b |
| Botrytis cinerea | | |
| Composition 2 + | 55 a | 90 a |
| Botrytis cinerea | | |
| Composition 1 | 0 c | 0 c |
| Composition 2 | 0 c | 0 c |
| None treatment | 0 c | 0 c |

As shown in Table 7, compositions of the present invention can be used to suppress weedy plants. More specifically, the treatment of the compositions 1 and 2 resulted in more severe occurrence of the disease to the tomato seedlings than the treatment only with Botrytis cinerea. However, in the case of using weed pathogens, weedy plants need to be defined specifically and the weed pathogens need to have high selectivity in pathogenesis. If not, weed pathogens may cause diseases to crops or plants because weeds are also plants. Lee et al. reported a similar result (Boyoung Lee, Dalsoo Kim, and Choong-Min Ryu. 2008. A super-absorbent polymer combination promotes bacterial aggressiveness uncoupled from the epiphytic population. Plant Pathology Journal 24:283-288). Inventors of the present invention showed that a composition of the present invention can improve aggressiveness of a bacterial plant pathogen to tomato seedlings.

### INDUSTRIAL APPLICABILITY

The present invention is industrially valuable in that it provides a composition that can supplement or replace chemical pesticides or chemical fertilizers and the use thereof.

1. A composition comprising SAP (super absorbent polymer) to improve biological activity or to increase microbial population of microorganisms that exist on surfaces.

2. The composition according to claim 1, characterized in that it additionally comprises at least one microbial nutrient that can be utilized by the microorganisms.

3. The composition according to claim 1, characterized in that it additionally comprises at least one beneficial microorganism that can inhibit growth of plant pathogens, harmful insects or weedy plants or can promote plant growth.

4. The composition according to claim 1, characterized in that it additionally comprises at least one beneficial microorganism that can inhibit growth of harmful animals.

5. The composition according to claim 1, characterized in that it additionally comprises at least one surfactant to improve spreadability of the composition on surfaces.

6. The composition according to claim 1, characterized in that it additionally comprises at least one single or complex mineral salt to improve wettability of the composition in water.

7. The composition according to claim 1, characterized in that said SAP is SAP made from starch.

8. The composition according to claim 1, characterized in that the content of SAP is the range of 20% to 95% by weight of the composition.

9. The composition according to claim 1, characterized in that the content of SAP is the range of 0.02% to 1.0% by weight per water volume when the composition is diluted with water.

10. The compositions according to claim 1, characterized in that said surface is selected from plant surfaces such as leaf, stem, branch or fruit surface, soil surfaces or animal skin surfaces.

11. The composition according to claim 2, characterized in that said microbial nutrients are composed of PLNT media that includes potato dextrose broth, Luria broth base, nutrient broth and tryptic soy broth each in the amount of 25% by weight based on their standard use amount.

12. The composition according to claim 3, characterized in that said plants are selected from the group consisting of cucumber, pepper, potato, rice, tomato, barley, wheat, pear and rose.

13. The composition according to claim 3, characterized in that said beneficial microorganisms are selected from the group consisting of genus Bacillus, genus Paenibacillus, genus Streptomyces, genus Trichoderma, genus Ampelomyces and genus Acremonium.

14. The composition according to claim 3, characterized in that said plant pathogens are selected from the group consisting of Magnaporthe grisea, Thanatephorus cucumeris, Phytophthora capsici, Botrytis cinerea, Puccinia graminis, Blumeria graminis (=Erysiphe graminis) and Sphaerotheca fusca.

15. The composition according to claim 3, characterized in that said harmful insects are selected from genus Plutella.

16. The composition according to claim 3, characterized in that said weedy plants are selected from plants that grow at time and place that plant growers do not desire.

17. The composition according to claim 4, characterized in that said harmful animals are selected from the group consisting of cockroach, ants and snails.

18. Use of the composition according to claim 1 on plant surfaces including leaf, stem, branch, flower or fruit, or soil surfaces on which lawn grasses grow.

19. Use of the composition according to claim 1 to inhibit harmful organisms on surfaces or to promote plant growth.
